(19) United States
(12) Patent Application Publication
(10) Pub. No.: US 2007/0073293 A1
(43) Pub. Date: Mar. 29, 2007

(54) System and method for flexible correction of bony motion segment

(76) Inventors: Erik O. Martz, Savage, MN (US); David Chow, West Chester, PA (US); Daniel Rosenthal, Short Hills, NJ (US); Timothy R. Miller, Holmdel, NJ (US); Jo-Wen Lin, Philadelphia, PA (US); Darrin N. Friend, Toms River, NJ (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/404,587

(22) Filed: Apr. 14, 2006

Related U.S. Application Data
(63) Continuation of application No. PCT/US04/34243, filed on Oct. 15, 2004.
(60) Provisional application No. 60/511,807, filed on Oct. 16, 2003.

(51) Int. Cl. A61F 2/30

(52) U.S. Cl. 606/61

(57) ABSTRACT

A non-rigid system and method for stabilizing displaced bony members include a flexible unit of tethering material coupled to the displaced bony members so as to restore a desired shape, curvature of or relationship between the bony members without excessively limiting mobility of bony members located adjacent to the displaced members the rest of the portions of the motion bony segment during a restoration process.

### CROSS-REFERENCE TO RELATED APPLICATION(S)

This application is a continuation of International Patent Application No. PCT/US2004/034243, filed on Oct. 15, 2004, which claims priority to U.S. Provisional Application No. 60/511,807, filed on Oct. 16, 2003, the contents of which are incorporated in their entirety by reference herein.

### FIELD OF THE INVENTION

The invention relates to a system and method for treating deformities and for reconstruction of soft tissue attachments of a mammalian skeleton. Particularly, the invention relates to a non-rigid system and method for stabilizing and balancing multiple bony motion segments. The invention also relates to a method of attaching autogenous or allogenic soft tissue to host bone for reconstruction of articular joint structures.

### BACKGROUND OF THE INVENTION

Skeletal deformities can have congenital and often hereditary causes and, if not treated, can result in severe health consequences. Among the numerous bones of a human or animal body, the spine, which is a flexuous and flexible column formed of a series of bones called vertebrae, is one of the most vital parts of the mammalian organism.

Ligament or tendon damage in the major articular joints is often caused by traumatic injury such as those seen in professional sports or motor vehicle accidents. The knee joint is comprised of the tibiofemoral and patellofemoral articular junctions and several major soft tissue attachments and is the most commonly reconstructed joint. It, along with other major articular joints, is critical to normal skeletal function.

Normally, the spinal column grows in line from the neck to the tailbone and, when viewed from the side, curves are seen in the neck, upper trunk, and lower trunk. The upper trunk has a gentle rounded contour called kyphosis and the lower trunk has a reverse of the rounded contour called lordosis. Certain amounts of cervical (neck) lordosis, thoracic (upper back) kyphosis, and lumbar (lower back) lordosis are normally present and are needed to maintain appropriate trunk balance over the pelvis. Deviations from this normal alignment may reflect abnormal kyphosis or lordosis when viewed from the side, or more commonly, scoliosis, when viewed from the anterior or posterior.

Under normal circumstances major joints consist of one or more articular junctions occurring between bony structures and several soft tissue (ligamentous and tendonous) attachments that are integral to motion and stability of the joint structure. Compromise of these soft tissue attachments results in partial to complete loss of joint function and stability.

Scoliosis is a sequential misalignment or deformity of the bones and discs of the spine and is manifested in the following ways. Firstly, the deformity can be an apparent side bending of the spine when viewed in a coronal plane from the front or back (anterior/posterior or AP view). Secondly, another way of diagnosing scoliosis is a loss of the normal kyphotic curvature in the thoracic or chest area when viewed from the side. This is a sagittal plane deformity. And thirdly, scoliosis can be observed as a result the rotation of the spine around its own long axis. This is an axial plane deformity. If scoliosis is left untreated, the curve can progress and eventually cause pain, significant cosmetic deformity, and heart, lung, or gastrointestinal problems.

Soft tissue damage leads to the loss of function, stability or alignment of the major articular joint structures and is diagnosed in the following manner. Firstly, physical examination of the joint and its motion characteristics may be performed to determine the extent of the loss of function and stability. Secondly, arthroscopic or radiographic, particularly MRI, methods may be used to further refine the physical diagnosis. Depending on the extent of the injury, some patients may function at an acceptable level without surgical intervention while others require major reconstruction to function reasonably well.

Treatment choices in scoliosis are determined by a complex equation, associated with the patient's physiologic maturity, curve magnitude and location, and its potential for progression. Treatment choices usually include bracing or surgery. Typically, the best treatment for each patient is based on the patient's age, how much more a patient is likely to grow, the degree and pattern of the curve, and the type of scoliosis.

The treatment choices for soft tissue injury are determined by a combination of patient activity level, age, physical health, extent of injury, and the likelihood of disease progression if the injury is left untreated.

The ultimate goal of treatment for scoliosis is the creation of desirable curvature in a portion of the spine. Some cases of scoliosis, if diagnosed at its earlier stages, can be managed without surgery. Otherwise, the curvature should be corrected by surgical procedures. Typically, a surgical procedure is associated with stainless steel or titanium rods affixed to the bone with hooks or screws, which then maintain the correction until fusion of multiple vertebral segments occurs. Surgery may be done from the front (anterior) of the spine or from the back (posterior) of the spine or both, depending on the type and location of the curve.

The treatment goal for soft tissue injury is to restore joint motion and stability to an acceptable functional level. A wide range of treatment options including surgical intervention may be used depending on clinical factors. Surgical treatment involves the repair or replacement of soft tissue elements with autologous or allogenic grafting materials fixed with screws, anchors or through biologic means. Surgery may be performed using open, minimally invasive, or arthroscopic methods. The surgical site and method are highly dependent on the location and extent of injury.

Overall, in addition to external bracing techniques, various surgical techniques are practiced to fuse the instrumented spinal segments. Some of the disadvantages and shortcomings of the surgery may include:
Poor or slow fusion rate;
Loss of segmental flexibility;
Loss of vertebral body height in the skeletally immature patients;
Poor self-image in adolescent patients who are braced for scoliosis;
Lack of curve stabilization; bracing is only successful in approximately 75% of patients;
As a result of multiple fusion surgical procedures for lengthening patients as they grow, a subsequent re-operation is as difficult as the original procedure and may require the removal or disablement of implants once a correction of spinal abnormalities is achieved;
A further consequence of multiple surgical operations and relative immobility of the fused spine may include the atrophy of the musculature; and
Children and adolescents, small in stature may not be physically able to tolerate the surgery required for a definitive fusion procedure.

Various surgical procedures are performed to treat soft tissue injuries. Disadvantages of surgical intervention include:
Unsuccessful treatment of the injury;
Post treatment pain; and
Cosmetic issues such as scaring.

To minimize at least some of the above-discussed disadvantages, it is known to use a cable system configured to maintain the desired position between multiple bones. U.S. Patent Application Publication No. 2003/0105459 discloses a stabilizing system including a plurality of inflexible cables each coupled to a respective fastener, which, in turn, is attached to a vertebral body. To generate a compressive force sufficient to maintain vertebral bodies in the desirable position, the free ends of the cables are coupled to one another.

In view of the aforementioned undesirable consequences posed by known rigid and non-rigid surgical systems directed to treatment for scoliosis, there is a need to provide a non-rigid system and technique for flexible correction of alignment between multiple bony portions, including spinal abnormalities producing significant curve correction, relative to one another while preserving much of the mobility of the bony portions to be fixed. This invention also can be used to reconstruct the soft-tissues surrounding major joints.

### SUMMARY OF THE INVENTION

The inventive system and method utilize flexible material to tether multi-segmental portions of a bony structure, such as vertebral bodies, finger and other limb portions, together while allowing certain mobility therebetween during a corrective process. As a result of corrective loads generated by the inventive non-rigid system, deformed bony portions tend to restore a desired curvature and/or shape.

Accordingly, unlike known surgical systems and techniques, the inventive system includes a less invasive and less traumatic procedure. Furthermore, in most cases, post-operative casting and bracing may not be required, leading to an expeditious discharge of a patient from the hospital, with a more rapid progressive resumption of routine daily activities. The inventive system and method allow for correction of abnormal curvatures of the spine while preserving its relative mobility, and flexibility, which, in turn, leads to sound muscle tone, less inconvenience and, overall, improved quality of the patient's life.

Unlike conventional systems, the inventive system utilizes allograft/autograft fascia material, which is easy to remove or disable. Furthermore, if made from bioabsorbable materials, the removal of the fascia material is not necessary once correction of scoliosis is achieved.

The inventive system may be successfully applied to not only treatment of minor degrees of spinal deformity, but also can be applied to more severe cases or other situations where restoration of natural curvature or dynamic fixation/stabilization is desired. For instance:
trauma—where instead of supplemental rigid fixation to vertebral body replacement devices, a less invasive flexible stabilization could be used;
interbody fusion products;
artificial discs; and
limbs and joint segments, such as fingers, toes, hand wrists, feet and ankles, which are deformed due to injury or disease, such as arthritis.
   Although the first two procedures may involve fusion, the use of flexible stabilization may be desired to reduce the rigidity of the spine above and below the fused segments. This has the advantage of better distributing the forces throughout the spine more uniformly and naturally. Whereas if a vertebral motion segment is made especially rigid, higher stresses may be seen in the adjacent flexible motion segments, potentially resulting in accelerated degeneration of the adjacent motion segments.

Flexibility of the inventive tethering system allows for its easy attachment to the spine by a variety of fasteners advantageously overcoming the complexity of the known systems. In accordance with one feature of the invention, the fascia material including at least one band can be directly attached to a posterior element of vertebra(e). In accordance with a further feature, the fascia material can be attached to the vertebrae by means of variously shaped and dimensioned fasteners.

In accordance with a further feature of the invention, the flexibility of the tethering system provides for equally effective treatment of a single vertebral motion segment (vertebra-disc-vertebra), multiple vertebral motion segments and motion segments constituting a finger or any other bony motion segment of a mammal body.

Therefore, in certain embodiments the present invention provides a tethering system for the flexible correction of spinal abnormalities, including scoliosis, allowing for a substantial degree of mobility of the spine over a period of treatment. The tethering system may have a simple and effective structure configured to minimize abnormal spinal curvatures extending over multiple vertebrae as well as to treat a deformed single vertebra. The tethering system may have a structure that can be effectively utilized in any of anterior, posterior and/or anterior/posterior-lateral surgical approaches. The tethering system may have a flexible structure configured to attach to various vertebral bony structures, such as spinous processes, lamina, facets, pars, pedicles, as well as the vertebral body itself, and vertebral bodies in a relatively simple and efficient manner.

In accordance with other embodiments, the invention further provides various innovative methods of configuring the tethering system.

Accordingly, the invention provides minimally invasive method for fusionless treatment of abnormal curvatures, or other deformities, of the spine while preserving much of the spine's flexibility during the treatment period, minimizing hospital stays, associated with reduced postoperative pain and less visible scarring, and improving the overall quality of the patient's life including a quicker return to school, work and other activities enjoyed before surgery.

### BRIEF DESCRIPTION OF THE DRAWINGS

FIG. 1 is a diagrammatic side view of a non-rigid system configured in accordance with the invention and attached to a portion of spine exhibiting an abnormal curvature;

FIG. 2 is a view of the non-rigid system of FIG. 1 and the treated portion of the spine shown to be exaggeratingly straight exclusively for the illustrative purposes;

FIG. 3 is a side view of the non-rigid system of FIGS. 1 and 2 attached to the spine in accordance with one embodiment of the invention;

FIG. 4 is a side view of the non-rigid system attached to the spine in accordance with another embodiment of the invention;

FIG. 5 illustrates the non-rigid system attached to the spine with a further embodiment of the invention;

FIGS. 6-8 illustrate some of various fasteners contemplated within the scope of this invention;

FIGS. 9-10 illustrate the inventive non-rigid system assembled in accordance with various embodiments of the invention;

FIGS. 11-14 illustrate the inventive non-rigid system attached to the vertebra(e) in accordance with various embodiments of the invention;

FIGS. 15-16 illustrate the inventive non-rigid system utilized in accordance with one of the embodiments of the inventive method and shown in pre and post treatment conditions of the spine, respectively;

FIG. 17 illustrates deformity extending over a few vertebral motion segments and a wedged shaped single vertebral motion segment, respectively;

FIG. 18 illustrates the inventive non-rigid system utilized to minimize the deformity shown in FIG. 17;

FIG. 19 illustrates the inventive non-rigid system configured and utilized with still another embodiment of the invention;

FIGS. 20 and 21 are side and front views, respectively, of a portion of spine treated in accordance with a further embodiment of the inventive system;

FIGS. 22 and 23 are front and side views, respectively, of a further embodiment of the inventive tethering system;

FIG. 24 illustrates still another embodiment of the inventive non-rigid system complemented with an auxiliary distracting system and method of utilizing the same;

FIGS. 25-28 illustrate various implementations of the auxiliary distraction system of FIG. 24;

FIGS. 29-33 illustrate further compositional and structural embodiments of the inventive non-rigid system;

FIGS. 34-37 illustrate still another embodiment of the inventive non-rigid system and the method for its implementation; and

FIGS. 38-40 illustrate the inventive method utilizing the inventive non-rigid system shown in FIGS. 29-37.

### DETAILED DESCRIPTION OF THE DRAWINGS

For the illustrative purposes, the following discussion will be mainly directed to the spine. It should be understood that the invention can be equally effective in treating deformities of fingers, toes, wrists, feet and other bony structures having at least one motion segment, which is composed of multiple bony portions.

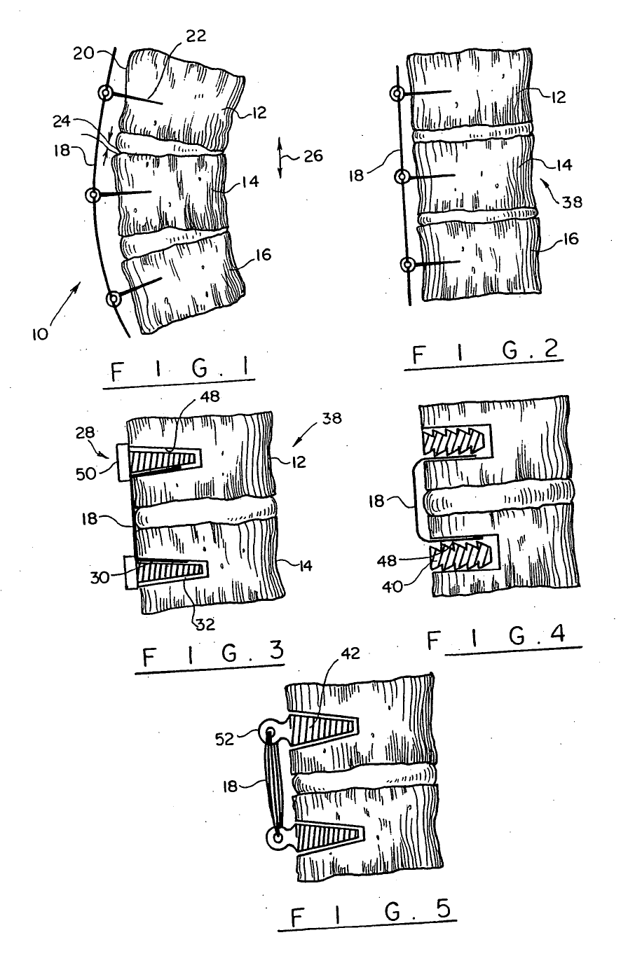

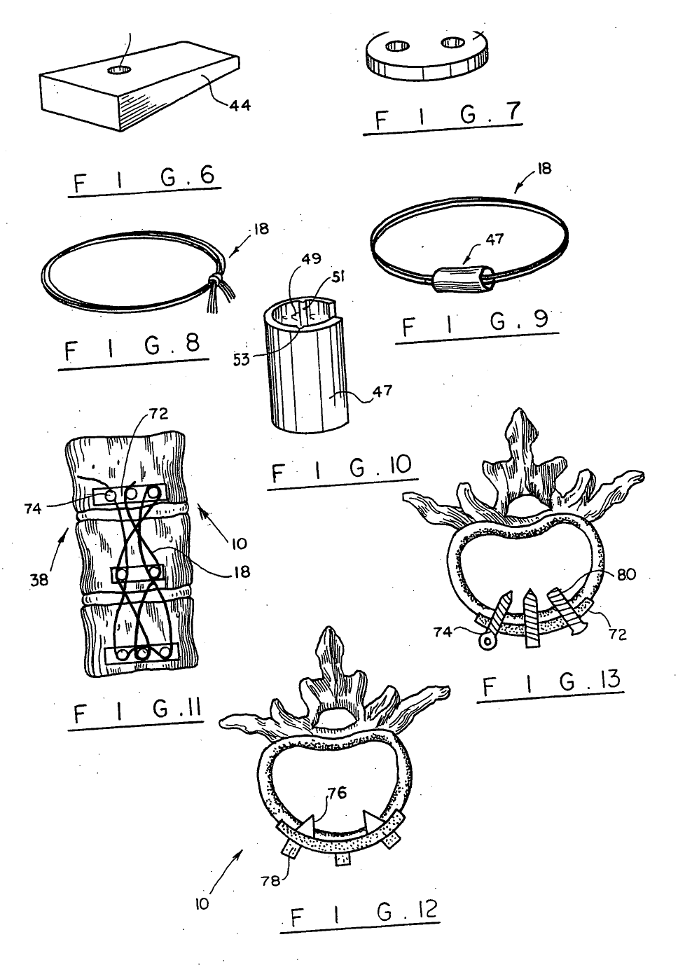

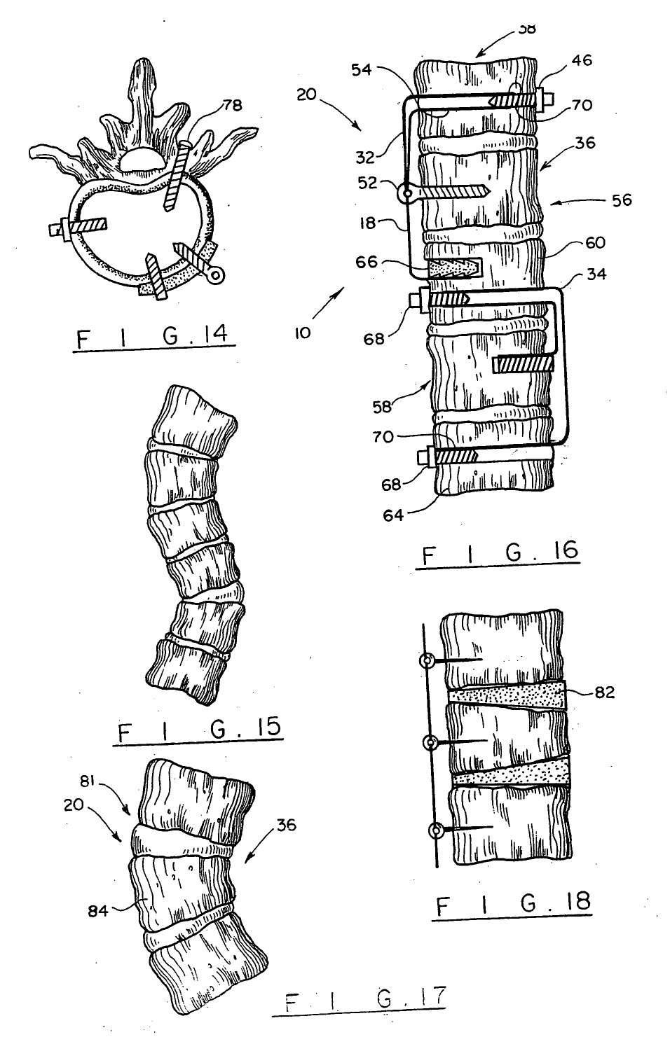

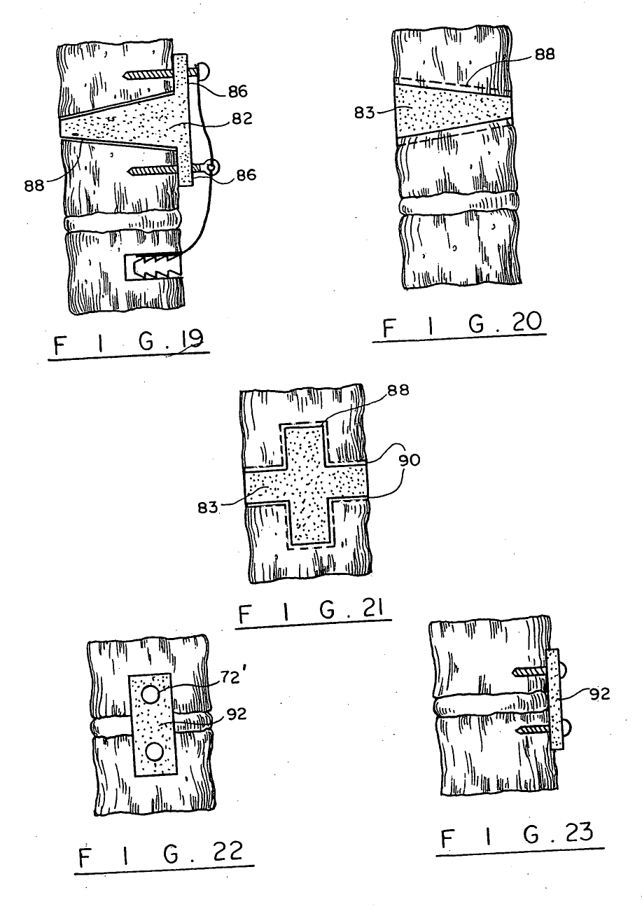

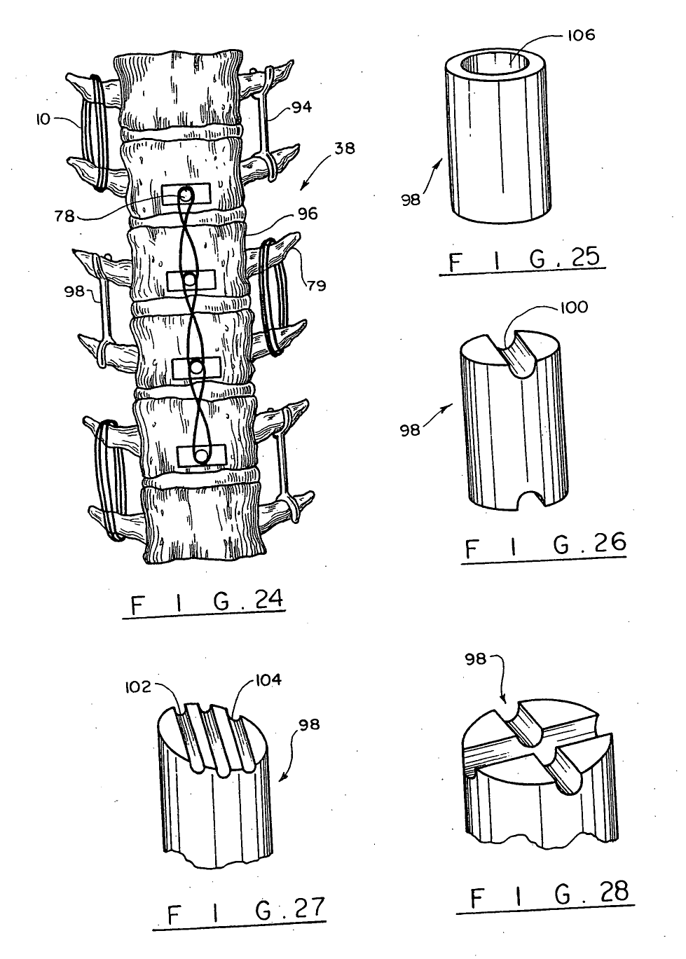

As shown in FIGS. 1 and 2, a flexible system 10 is utilized to treat scoliosis affecting vertebrae 12, 14, and 16, which have exaggerated convexity in a frontal (posterior/anterior) plane. The system 10 includes at least one flexible piece including, in accordance with one embodiment, flexible material 18 attached across several vertebral motion segments or a single vertebral motion segment, so as to apply corrective loads including tensile, compressive, rotational, or a combination thereof. As a result, the inventive system 10 prevents spinal deformity progression and subsequently minimizes or fully corrects it without having adjacent vertebrae fused, which allows the spine to remain flexible, yet stable.

The tethering material may be made from fascia, which, as a term used in this disclosure, describes a single segment, length, piece, etc of tissue capable of maintaining the corrective loads between at least two bony members. As is known, the fascia extends under the skin to cover underlying tissues and to separate different layers of tissue. Accordingly, the flexible material 18, when comprising fascia, can be obtained from the patient's body and, in this case, be characterized as an “autograft” fascia. Alternatively, fascia may be obtained from a foreign body or material and, in this case, be termed as an “allograft” fascia. Structurally, the tethering material can include multiple pieces, bands, or loops, or a single continuous piece or loop.

The flexible material 18 (or tethering material) may comprise materials other than fascia. Other alternatives include fabrication of the tethering material in whole or in part from biocompatible fibers of a native, biosynthetic, or synthetic polymeric, connective tissue or plant connective tissue-like characterized by the biocompatibility of the selected material. The tethering material may be resorbable or degradable to eliminate the necessity of the secondary operation directed exclusively to the removal of the tethering material once the correction is achieved. Alternatively, the tethering material can comprise non-resorbable polymers, metals, etc., similar to a flexible wire or cable. Overall, the tethering material can include abdominal peritoneum, tendons, small intestine submucosa, perichondrial tissue, completely or partially demineralized bone, ligament, silk, collagen, elastin, reticulin, cellulose, and a combination thereof.

The physical properties of the tethering material 18 such as length and number of pieces, loops or segments, which may or may not be braided, like a rope, or be tied together, are selected to generate a predetermined sufficient force. As a result, the tethering material may be pre-packaged in discrete lengths, loops or segments, various thickness/diameters, sizes, etc., so that the surgeon does not need to assemble these at surgery. Accordingly, in one embodiment, a packaging for pre-packaged tethering components may be rated for various loads, for example, a number 1 package could maintain tensile loads of up to X newtons, and a number 2 package could maintain tensile loads of up to 2× newtons, etc. This would allow the surgeon to apply the correct type of tethering material after determining the necessary load, which is applied to multiple bony members for initial restoration of the desirable relationship therebetween, as explained herein below in regard to the inventive method.

Generally, the surgery for idiopathic scoliosis includes initial segmental derotation to correct segmental curvature by using compression or distractor instruments. The surgeon may elect to use traditional metal hook and screw systems to temporarily correct the spinal deformity while the tethering system is applied, and then remove the traditional hardware prior to closing the wound. An initial load may be determined by a compressor/distractor instrument that has a force gauge associated with it, such as a calibrated spring. The determined load, which is typically a tensile load, should be sufficient to dislocate bony members relative to one another so as to restore the desired curvature, shape or relationship therebetween and an intervertebral disc of a single motion segment or multiple motion segments.

Having restored the desirable relationship, the surgeon couples the inventive non-rigid system, which has predetermined physical qualities, to the loaded bony members. If the load is tensile, the corrective force generated by the inventive system is compressive and sufficient to maintain the desired load during a healing period upon removal of the compression/distraction instrument(s) or implant(s) used for the application of the desired load.

FIG. 2 represents the surgical stage characterized by the applied tethering system 10, as illustrated in FIG. 1, which provides for applying compressive forces 24 (FIG. 1) to the vertebrae 12-16 performing the compression of their lateral portions 20, since most scoliosis, but not all, is deformation in a medial/lateral plane, and the decompression of the opposite medial portions, as indicated by arrows 26 (FIG. 1). A tensile load borne by the inventive system 10 is a function of the maintenance of the compressive load to the left side of the spine, as seen in FIGS. 1 and 2, and may vary depending on the composition and structure of the material 18. As a result of the inventive procedure, the curvature can be normalized in accordance with normal shape, as shown in FIG. 2.

Any suitable fasteners 22 may be used to couple the tethering material 18 to the spine and may include screws 28 (FIG. 3), wedges 40 and 44 (FIGS. 4 and 6), suture anchors (FIG. 5) wedge buttons 46 (FIG. 7), clips, snaps, other friction fittings, compressive fittings, expanding rivets, staples, nails, adhesives, etc. The fasteners may be solid or hollow, the latter allowing the tethering material to be looped through the fastener, or for supplemental fusion or healing material to be placed. Materials used for manufacturing the fasteners include metal, shape memory materials (such as Nitinol), carbon graphite composites, ceramics, polymers, and others, any of which may be biodegradable, resorbable, or non-bio-degradable. These materials may or may not be used in combination with allograft bone, autograft bone, xenograft bone, bone powder, bone particles, or bone fibers. Bone fusion enhancing substances, for example bone morphogenic proteins (BMPs), DNA vectors expressing BMPs, or bone fusion enhancing processes, such as surface demineralization of the bone, may be added to or performed on the above-discussed materials and parts to enhance or increase fixation to bone.

While the description mainly relates to a structure of flexible system, the scope of the invention encompasses its broad application. For example, one embodiment comprises a surgical kit comprising a plurality of components of the flexible system and may include a rigid fixing system. Accordingly, the kit may include one or more fasteners, fastener inserter(s), such as a driver, drill and the like, length(s) of tethering material, a compressor/distractor instrument that has a force gauge, such as a calibrated spring, and any other component, as disclosed within the scope of this invention.

Along with variously configured fasteners, attachment of the tethering material 18 to the spine 38 may be accomplished by a variety of techniques. For instance, FIG. 3 illustrates an interference technique in which the tethering material 18 is pressed between a textured shank 30 of the screw 28 and a surface 48 formed within a vertebral body upon driving the screw 28 into each of the vertebrae 12-14. Additionally, the head 50 of the screw 28 may be dimensioned to overlap an opening formed in the vertebral body as result of the screw's introduction and, as a consequence, press the tethering material 18 towards the outer surface of the vertebra. To enhance the attachment of the tethering material to the spine 38, the tethering material 18 can be looped around the shank of the screw 28. Wedge/blocks 40 (FIG. 4) and wedges 44 (FIG. 6) can also be utilized to carry out the interference technique by being pressed into the vertebral bodies, or into the vertebral disc space, so as to form a nest therein while urging the tethering material 18 against the nest's inner surface 48. The wedges 40 and 44 each may be provided with a channel(s) or aperture(s) 45 traversing the body of the wedge and configured to serve as an anchoring structure receiving the tethering material 18. Alternatively, the wedge can be used as a fusible implant configured to sculpture a defected vertebral body or disc space, as will be discussed below. In this case, the aperture 45 serves as a receptacle into which either fusion promoting material such as demineralized bone matrix can be placed, or through which the tethering material can be passed through, or a combination thereof. The aperture(s) can be formed within any convenient region or regions of the wedge 40, 44 to address the specific requirements imposed by any given procedure.

The tethering material 18 can be secured directly to variously shaped fasteners instead of relying on the interference between the fastener and the vertebral body. For example, FIG. 5 illustrates suture anchors 42 each having an outer end 52, which is coupled to the tethering material. As shown in FIG. 5, the outer end 52 may have an eyelet portion configured so that the tethering material can be tied, knotted (FIG. 8), glued, welded, clamped, crimped, or otherwise coupled to the anchor. FIGS. 9 and 10, in turn, illustrate a coupler 47 configured to secure opposite ends of the tethering material 18. The inner surface of the coupler 47 can have formations 49, such as spikes, grooves, barbs, ridges, knurling, etc., which are configured to engage the tethering material 18. The structure of the coupler 47 may be malleable or deformable and can be made from plastic, a thin metal sheet, or other material to conform to the desired shape necessary to lock or secure the ends of the tethering material together. In addition or alternatively to the formations 49, the coupler 47 may have a projection 51 and a key hole 53 lockingly engaging one another and the tethering material after the coupler has been deformed. The actual position of the projection and key hole system may vary and need not be on the same side of the coupler, for example projection 51 could engage a key hole (not shown) on the outer surface of coupler 47, resulting in a slight overlap upon engagement.

The flexibility of the inventive system 10 allows the fasteners to be selectively mounted to different posterior, anterior lateral and medial regions of the spine. For instance, as shown in FIG. 16, two separate segments 32 and 34 of the tethering material 18 each are attached to a respective group 56, 58 of three consecutive vertebrae to apply oppositely directed compressive forces. To provide the desired correction, a construct has a middle vertebra 60 common to both groups 56, 58 and provided with two fasteners 66, 68 attached to the segments 32 and 34, respectively, whereas the rest of the vertebrae each has a single fastener. In the embodiment shown, free ends 70 of the bands 32, 34 each extend through the entire vertebral body of the respective end vertebrae 62, 64 and are either attached to the sides of vertebrae opposite to the sides of entry of these bands by the fasteners 68.

Alternatively, the bands can be looped around a fastener so that the segment(s) of the tethering material are further pulled back and attached to other vertebral segments in any convenient manner. For instance, the free end 70 of one of the tethering segments can be looped through the button 46 affixed to the side of the vertebrae opposite the entry point. Depending on the local requirements, the free end 70 can be knotted or otherwise secured the suture anchor 52 or to any of desired vertebral bodies of the spine 38 by the wedge 66. A variety of attachment arrangements is limitless subject only to a number and configuration of the fasteners and, of course, to the specifics of a given procedure.

This embodiment is also illustrative of a number of the segments constituting the tethering material 18. Thus, the end vertebrae 62, 64 each are connected to a respective inner vertebra, located immediately next to it, by, for example, a two-band tethering material, whereas the rest of the inner vertebrae, which would experience lower loads, can be interconnected by a single-band tethering material. Single fasteners can support multiple tethering segments.

Referring to FIGS. 11-13, the inventive system 10 can be further configured to have tethering posts providing for the attachment of the tethering material 18 to the spine 38. In one embodiment, the tethering post includes a support plate 72 in combination with a fastener and is configured to be applied to a single portion or multiple portions. Numerous types of fastener can be used including suture anchors 74, crimps, screws, nails, pins, and/or other fasteners connected to the vertebrae. Advantageously, multiple support points provided by the fasteners allow the tethering material 18 to be attached in a variety of configurations, including, for example, a shoelace pattern (FIGS. 11, 24, 36), a criss-cross pattern, repeating loops, a linear array, or a mesh-pattern. The shoelacing can be done through or around the suture anchors 74 or 78.

Any of the aforementioned fasteners may be used in conjunction with the tethering post. Alternatively, or additionally, the support plate 72 may have an inner side carrying a plurality of spikes 76 formed integrally with the element, as shown in FIG. 12. Accordingly, the unitary plate 72 may be spiked into the vertebral body without the use of fasteners. In one embodiment, such unitary tethering post may be made from a shape memory metal, whose spikes, once inserted into the vertebrae, would deform to resist movement of the plate or post. Alternatively, as shown in FIG. 13, the plate 72 can be screwed to a single vertebral body with distal ends of screws 80 having proximal ends acting as posts or loops for the tethers.

Another embodiment of the flexible system 10 is shown in FIGS. 22 and 23 and includes the flexible piece configured as a plate-like element 72′. Conceptually, the element 72′ should sufficiently flexible to apply and maintain a corrective load to at least two portions or multiple vertebral bodies similarly to the other types of tethering material 18. The element 72′ may be made of bone material. If made from bone material, the bone material may be completely demineralized or segmentally demineralized, for example in a middle region 92, as shown in FIGS. 22 and 23 to improve the flexibility of the plate. Thus, the element 72′ can be sufficiently flexible to produce corrective loads sufficient to minimize or eliminate the deformity, and/or correct a shape without the use of tethering material. Materials such as metals, polymers (resorbable or non-resorbable), ceramics, composites materials (polymer/metal, polymer/bone), or others may also be used for the element 72′.

Note that any combination or pattern of the fasteners and posts can be utilized in combination with both plate-like element 72′ and support plate 72 to meet the specific surgical requirements of the patients. Thus, as illustrated in FIG. 14, the tethering posts 78 can be applied to any of anterior, posterior, lateral and medial portions of the vertebral body or any of the posterior elements, just as the aforementioned fasteners. Posts could have flared ends to enhance tether securement. Furthermore, the tethering system can have multiple pieces of tethering material 32 and 34, as illustrated in FIGS. 15 and 16, attached to the lateral 20 and medial 36 sides of the spine 38 to correct multiple abnormal curvatures. Accordingly, the inventive tethering system 10 can be equally effective when applied to a single vertebra or multiple vertebrae subject only to the number and length of the pieces of lengths constituting the flexible piece of the system.

Often times in order to correct severe scoliosis, the shape of the vertebral body itself may need to be corrected or restored to a more natural shape. For instance, sometimes the frontal profile of the vertebral body in scoliosis patients are wedge shaped 84 (FIG. 17), not square or rectangle as with a normal spine, which help contribute to the abnormal curvature of the spine. In this case, the flexibility of the inventive tethering system 10 allows for tethering to be done in combination with constructs configured to correct such a bodily defect. To realize the latter, another wedge piece 82 (FIG. 18) could be either added to or could replace the original intervertebral disc or added directly to the vertebral body itself, and, in combination with the tethering, to correct the abnormal curvature. Despite the fact that the added wedge 82 can fuse the adjacent vertebral bodies, some degree of flexibility can still be maintained because the entire length of spinal segments affected by the abnormal curvature would not be fused. Therefore, an overall amount of flexibility would be preserved. In this case, the tethering system 10 can be additionally used to enhance fusion between the fusible parts of the damaged vertebra by having its opposite ends attached to these parts. In conjunction with this embodiment, the wedge 82 can be configured similarly to either one of the wedges 40, 44 (FIGS. 4 and 6) and be particularly advantageous by serving simultaneously as a support for the tethering material 18 and as a fusible implant. Also, the plate-like element 72′, as discussed above, can be used to extend over the wedge 82, which may be specifically configured to restore the desired shape of the severely deformed motion segment. Accordingly, the plate may function as a supporting barrier preventing expulsion of the implant from an implant site.

Alternatively, the deformity illustrated in FIG. 17 could be corrected using appropriately sized and shaped artificial intervertebral disc implants, and the tethering system could be applied to aid in restoration and maintenance of the stability of this system. Such artificial disc implants are typically implanted into the prepared intervertebral disc space, and will restore flexibility to this motion segment, as opposed to an intervertebral fusion implant, which will fuse the motion segment.

The added wedge 82 could be demineralized partially on the outer surfaces to enhance a fusion process and to impart a slight degree of flexibility, even if the juxtaposed surface of the end plate fuses to the adjacent vertebral bodies. Alternatively, the wedge can be completely demineralized. As shown in FIG. 18, the wedge 82 may be configured to extend through the entire intervertebral width or only through a portion of the adjacent vertebral bodies and, thus, can grow into the entire contact surface or only the selective portions thereof.

Universality of the inventive system 10 as a corrective system and as a support for implants allows it to be a viable alternative to a rigid fixation system, which typically includes multiple pedicle screws, rods, hooks, anterior plates and/or anterior screws, or the two can be combined to supplement one another. Thus, when the deformation of a single spinal segment (vertebra-disc-vertebra) is severe enough to have the surgeon consider the use of the rigid fixation system, only for example, an apex of the curvature can be fused and corrected/derotated by means of the rigid system. The rest of the vertebral segments contributing to lesser extents to the deformed spinal curvature, located adjacent or spaced from the vertebral motion segment to be fused and stabilized via said rigid fixation systems, can be corrected/derotated via the flexible stabilization by the inventive tethering system 10, if needed. Note that the flexible tethering material 18 can be configured to have a selective number of intertwined lengths thereof to provide the desired thickness/strength of the material, which would be sufficient to generate various corrective loads as well to ensure the desired position of the implant.

Accordingly, any of the tethering systems described herein can be used as a stabilizer/barrier to expulsion for interbody fusion procedures that in addition to or as an alternative to the correction of deformities, may simply be used to restore disc height in order to relieve pain. In this case, the tethering system 10 would bridge the disc space preventing the expulsion of the implant therefrom while providing stability between the coupled vertebrae. Securement of the implant in the disc space can be significantly enhanced by providing the tethering system 10 with numerous lengths of the tethering material 18, which can be braided, netted, intertwined, interwoven, tied together, to form a reliable barrier capable of preventing the displacement of the implant.

To improve the structural strength of the tethering system 10, the added wedge 82 can have laterally extending arms 86 made integral to the tethering posts, as seen in FIG. 19. Alternatively, the cross-shaped fastener/implant 83 can be directly introduced into channel 88 cut within the opposing end plates 90 of the adjacent vertebrae to enhance stabilization of the fastener 83, as illustrated in FIGS. 20 and 21. Stability of a spinal segment exposed to corrective loads produced by the inventive tethering system 10 can be improved by utilizing the latter with a distraction system 94, as shown in FIG. 24. The system is configured to prevent the reverse displacement of the vertebrae under the corrective load generated by the tethering system 10 and, preferably, to be applied segmentally along a portion of curvature across multiple vertebral levels.

As seen in FIG. 24, the distraction system 94 can be applied directly to transverse processes or other posterior spinal elements of the vertebral body 96 or therebetween or between the disc spaces, while the tethering system 10 is applied to the other portions of the vertebral bodies. The distraction system 94 is configured to generate the desirable load applied along the concave side of the spine, while the tethering system 10 generates compressive forces, which combined with the tensile load of the system 94, tend to stabilize and balance the coupled vertebrae. The distraction system could be used intermittently throughout the long length construct including multiple vertebrae. As an alternative to the posterior elements, the distraction system can be utilized with the fasteners and attaching means discussed previously. For example, the distraction system may be engaged between the tethering posts simultaneously used by the tethering system 10. Alternatively, such a distracting system could also be applied to projections 74 of support plates 72, to apply destructive forces. Such plates/tethering posts 72 could wrap around the vertebral body (lateral-anterior-lateral) so that different segments of the plate/post could support compressive tethering or distractive elements. Any of the posterior elements of the spine 38 can be used as a tethering post if the operating surgeon would find such use appropriate. The distracting system adds stability and balance to the tethered vertebrae. While in some situations, no contouring of the posterior elements is necessary, other situations may require their shaping.

The distracting system 94 may include variously configured shafts 98 made from pieces of cortical bone that may constitute either the entire shaft lengths of the system 94, or sections thereof. These sections may be sectioned parallel, perpendicular, or at an angle to the long axis of the cortical bone shaft. A structure of the shafts 98 is designed to facilitate the attachment of the shaft to a shaft supporting structure, which, depending on the location of any given shaft, may be any of the posterior elements of the vertebral body or previously tethering posts, such as 78. As shown in FIGS. 25 and 26, the shaft 98 can have a central recess 106 on its end to better engage the posterior elements or the posts or a central notch 100. Recess 106 could mate with a formed protrusion on a portion of the vertebrae, made by the surgeon to better engage the shaft 98. Notches could be “V”, “U”, or “L” shaped, with multiple notches and/or intersecting over, to allow easier insertion onto the tethering posts or to better match patient anatomy if the shafts 98 are mounted to the posterior elements. Alternatively, taking into account the geometry of the posterior elements, such as spinous processes, lamina, facets, pars, pedicles, the shaft 98, as shown in FIG. 27, is structured to have multileveled notches 102, 104 capable of reliably engaging the elements and formed in a plane, which, in general, extends angularly at an angle a with respect to a normal N-N to the central spine axis.

Yet a further modification of the shafts 98 includes a plurality of intersecting grooves, as illustrated in FIG. 28. Shafts 98 can be made from bone, bone composites, polymers, ceramics, metals, etc. The surfaces of notches 100, 102, 104 can optionally be roughened to improve fixation. Roughening can include spikes, pyramidal protrusions, grooves, splines, etc. Additionally, the surfaces can be demineralized, alone or in combination with the surface roughening.

As with the fasteners, couplers, anchors, screws, etc., the shafts can be treated with substances to stimulate bony fusion as well as prevent infections. Additionally, the flexible members can be treated, coated, prepared with a substance or substances that will inhibit scar formation, fusion, or prevent infections. Phytochemical compounds have inhibitory effects on keloid fibroblasts (KF) and hypertrophic scar-derived fibroblasts (HSF). Compounds such as, hydroxybenzoics, flavonols [i.e. quercetin and kaempferol], and turmeric curcuminare are potential scar inhibitors. These hytochemicals inhibit fibroblast proliferation by inducing cell growth arrest but not apoptosis. The compounds quercetin, gallic acid, protocatechuic acid, and chlorogenic acid are the strongest inhibitors. Tamoxifin, 5-fluorouracil, matrix metalloproteinase inhibitors and TGF-Beta inhibitors can also reduce postoperative scarring. It has also been shown that the use of external irradiation, Agaricus bisporus (edible mushroom lectin), tetrandrine, and chitosan-polyvinyl pyrrolidone hydrogels may be effective scar inhibitors.

The inventive system may be used as a flexible, or non-flexible “bridge” between any of the posterior processes of two or more vertebrae. The system can be attached to the (posterior) spinous, transverse, mammillary, and articular processes as well as to the pedicles or the lamina using screws or snaps, or could even slip around several processes like a rubberband or a cap and then be secured with screw, pins or snaps. Such as bridge can be made of either bone or a compatible synthetic material, as disclosed above. It may be treated to prevent bone growth in the case where union is not desired. The bridge can alternatively enhance fusion to the spinous, transverse, mammillary, and articular processes as well as the pedicles or the lamina.

Another embodiment of the tethering system 10 may include a construct configured of a tethering material, which is formed naturally and integrally with the end bone segments shaved to have the desired shape and be used as fasteners. Compositionally, this system includes, for example, Bone-Tendon-Bone (BTB) portions of tissue.

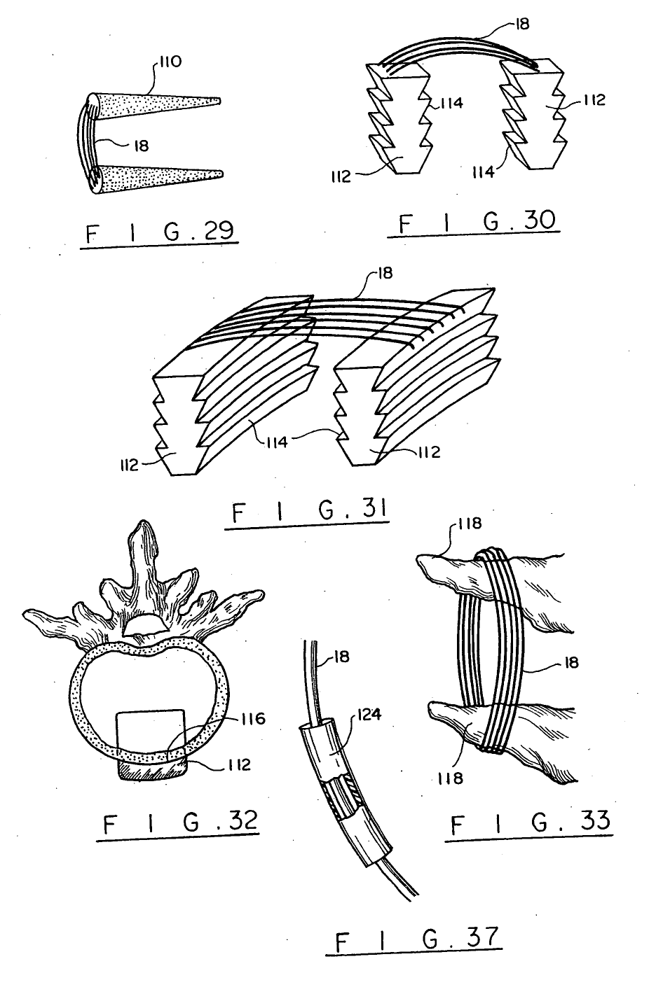

As illustrated in FIGS. 29-31, fasteners made from bones and representing the above-disclosed BTB tethering system can be variously shaved. For instance, FIG. 29 illustrates fasteners 110 configured as suture anchors. Alternatively, as illustrated in FIG. 30, the opposite ends 112 can be wedged-shaped and have ridges 114 formed on the opposite surfaces of each wedge. The formations of the ridges on each wedge can be arranged uniformly including the same orientation and pattern on the opposite sides of the wedge. Furthermore, each of the opposite sides can have a unique orientation and pattern. Also, two (or more) wedges constituting the tethering system can have respective surfaces provided with uniformly oriented and patterned ridges or any of these surfaces may be uniquely textured. Even the same side of each wedge may have regions with differently oriented and patterned threads.

While each of the configurations of the tethering system 10 is illustrated as having uniformly shaped fasteners, the latter may have different shapes and cross sections. Thus, for example, at least one of the wedges can have a square cross section (FIG. 30) or a rectangular cross section, as illustrated in FIG. 31, whereas the other one can be a screw, a button or any other differently shaped fastener. Note, if both end fasteners are threaded, the flexible tethering material 18 might get twisted during insertion, but this may not adversely affect performance. To better conform to the contours of the vertebral bodies, the fasteners 112 may have arcuate demineralized surfaces 116 juxtaposed with the vertebral bodies, as can be seen in FIG. 32.

Also, instead of the fasteners, the transverse and/or posterior processes 118 can be used as tethering posts, either naturally, or by contouring them for supporting the tethering material 18 looped over the fasteners' outer ends. Any of the posterior elements such as pedicles and any of costal, mammilary, accessory, inferior, superior processes and spinal processes, as shown in FIG. 33, can be used to support the tethering system 10.

Still a further embodiment of the tethering system 10 includes only a bone structure, which has selectively demineralized portions serving as a flexible tethering material, which is capable of supporting tensile loads. In particular, consecutive portions of the bone may be selectively (segmentally) demineralized (SDB) to provide at least one intermediary flexible portion, whereas the respective end portions remain mineralized and serve as fasteners, which can feature any desired shape. While the length of the demineralized intermediary portion can vary, it may be advantageous to demineralize about ⅓ of the entire length. As can be readily understood, all disclosed embodiments of the inventive tethering system 10, including the bone-tendon-bone (BTB) and segmentally demineralized bone (SDB), can be used to join adjacent or non-adjacent motion segments. Alternatively, such a bone segment can be segmentally demineralized in multiple places, which would advantageously be used to span multiple vertebral motion segments.

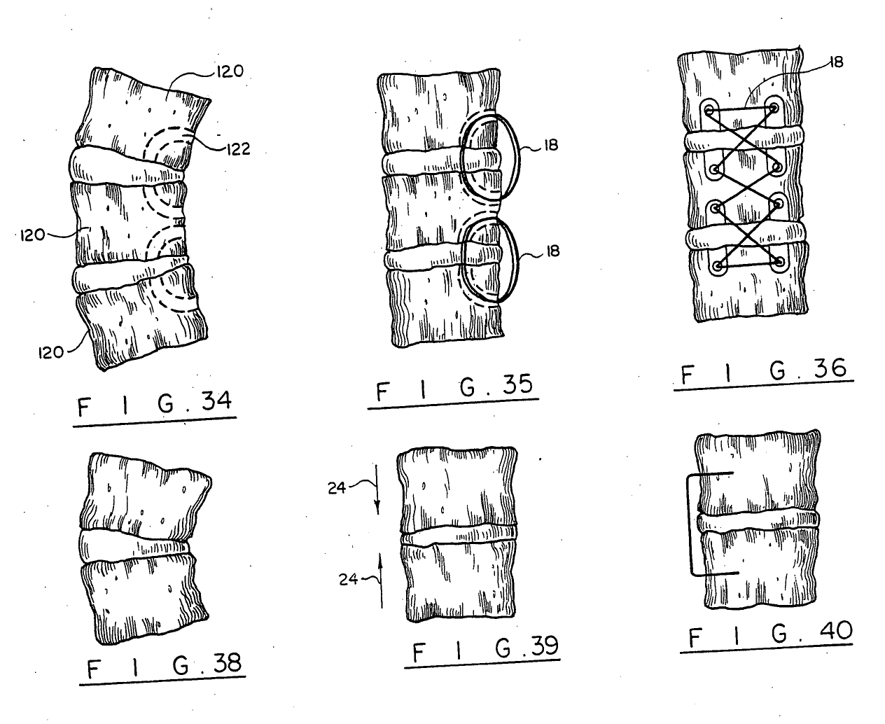

Vertebral bodies 120 can have channels or tunnels 122 (FIGS. 34-36) cut into them to accept the tethering material 18 which extends through the channels to form at least one loop or to interlace the vertebral bodies in a shoe-lacing pattern, as seen in FIG. 36. The loops can be prefabricated so as to have any desired size or thickness. Thus, one or more lengths of tethering material may be formed to apply the desired corrective load upon assessing the latter. A single piece of tethering material can be preformed into a single or multiple loops; alternatively, multiple pieces can be preformed into single or multiple loops. These loops can be formed by knotting, gluing, crimping, welding, chemically bonding, molding, or other means of securement. To decrease any stress concentration in the tethering material, protective sleeves 124 (FIG. 37) can be applied between the tethering material 18 and the surface of the channels 122 within the vertebral bodies. The sleeves 124 can be pre-loaded onto a line of tethering material fascia or applied during surgery. Additionally, the sleeves can have shoulders configured to abut the outer side of the vertebral body. Such shoulders can have protrusions or surface roughenings extending towards and reliably engaging the vertebral body. The configuration illustrated in FIGS. 34-36 allows the tethering system to effectively treat deformities by utilizing a single fastening element, be it one of the natural processes or any artificial fastener, or without using a fastener. Returning to FIG. 35, the tethering material 18 can be guided through channels 122 formed in one of or multiple vertebrae and, upon applying a compressive force to opposite ends of the material 18, the latter can be reliably locked into the desired position.

A method of affixing any of the described tethering systems 10, including but not limited to the bone-tendon-bone (BTB) or sequentially demineralized bone (SDB), is illustrated in FIGS. 38-40. Initially, the segments to be treated by the tethering system 10 are controllably loaded upon determining the desirable load via available surgical instruments or implants that may be inserted temporarily (FIGS. 38, 39), as indicated by arrows 24, to restore a normal curvature. The tethering system is installed by inserting the opposite ends thereof into the vertebral bodies, as illustrated in FIG. 40 so that it generates a compressive force directed to compress/decompress the segments upon releasing the initial compressive force that was applied through the instruments or implants.

It will be understood that various modifications may be made to the embodiments disclosed herein. Furthermore, the inventive tethering system, described primarily in the context of the spine curvatures, can be equally effective in treating any other bony motion segment including two or more relatively displaceable portions. Therefore, the above description should not be construed as limiting, but merely as exemplifications of preferred embodiments. Those skilled in the art will envision other modifications within the scope and spirit of the claims appended hereto.

1. A system for stabilizing a plurality of bony members relative to one another, the system comprising a flexible unit selectively coupled to the plurality of bony members and configured to have a predetermined length, thickness, quantity and a size to generate a corrective force applied to and sufficient to stabilize the coupled bony members upon establishing a desirable relationship therebetween.

2. The system of claim 1, wherein the flexible unit has a polygonal or circular cross-section and is made from at least one length of material selected from the group consisting of fascia, abdominal peritoneum, tendons, gracilis, iliotibial band, small intestine submucosa, perichondrial tissue, completely demineralized bone, partially demineralized bone, ligament, silk, collagen, elastin, reticulin, cellulose, and a combination thereof.

3. The system of claim 2, wherein the fascia, abdominal peritoneum, tendons, gracilis, iliotibial band, small intestine submucosa, perichondrial tissue, completely demineralized bone, partially demineralized bone each are made from a piece consisting of an autograft flexible unit, an alograft flexible unit, a xenograft flexible unit, and a combination thereof.

4. The system of claim 2, further comprising at least one first fastener attached to one of the coupled bony members and operatively connected to the flexible unit.

5. The system of claim 4, wherein the at least one first fastener is directly attached to the flexible unit permanently or detachably, the flexible unit being coupled to the one and at least one second bony members and tensioned therebetween to generate the corrective force selected from the group consisting of compressive force, tensile force and a combination thereof and sufficient to maintain the desirable relationship between the coupled bony members.

6. The system of claim 5, wherein the at least one first fastener is selected from the group consisting of screws, wedges, suture anchors, wedge buttons, clips, snaps, friction fittings, compressive fittings, expanding rivets, staples, nails, crimps and a combination thereof, the system further comprising at least one second fastener.

7. The system of claim 6, wherein the one and at least one second fasteners and the flexible unit are configured to define a naturally formed unitary body, the flexible unit being more flexible than the first and second fasteners.

8. The system of claim 7, wherein the naturally formed unitary body includes Bone-Tendon-Bone (BTB) portions of tissue, wherein the tendon constitutes the flexible unit and the bones each form a respective one of the fasteners.

9. The system of claim 6, wherein the flexible unit includes a plate-like element extending between the one and at least one second bony member and having opposites ends coupled to the first and at least one second fasteners, respectively, extending into the one and at least one second bony members.

10. The system of claim 4, further comprising a support plate coupled to the at least one first fastener to form a tethering post for engaging the flexible unit, the support plate having a smooth or textured bearing surface, which is juxtaposed with an outer surface of the one bony member, and being configured to conform to the outer surface of the one bony member.

11. The system of claim 10, wherein the support plate and the at least one first fastener are detachably coupled to one another or fixed permanently to one another, the support plate being made from material selected from the croup consisting of metals, shape memory alloys, carbon graphite composites, ceramics, polymers, hygroscopic material, allograft bones, autograft bones, xenograft bones, bone powder, bone particles, bone fibers and a combination thereof.

12. The system of claim 10, wherein the tethering post is either detachably attached to the one bony member and is capable of being removed with the flexible unit, or permanently attached to the one bony member, the support plate having an outer surface provided with protrusions spaced from the at least one fastener and configured to be coupled to the flexible unit.

13. The system of claim 10, further comprising a second fastener, the at least one first and second fasteners extending through the support plate and each having a respective distal end introduced into the one bony member.

14. The system of claim 13, wherein the support plate and at least one of the first and second fasteners are integrally coupled to form a one-piece body, the bearing surface of the support plate having at least one protrusion extending into the one bony member.

15. The system of claim 6, wherein the at least one first and second fasteners each have a distal end provided with a smooth or textured surface configured to be introduced into the one bony member so as to form a nest and to press a portion of the flexible unit against a wall of the nest.

16. The system of claim 6, wherein the at least one first fastener is made from material selected from group consisting of biodegradable material, resorbable material, non-resorbable material, non-biodegradable material and a combination thereof.

17. The system of claim 6, wherein the at least one first fastener is made from material selected from the group consisting of metals, shape memory alloys, carbon graphite composites, polymers, ceramics, hygroscopic material, allograft bones, autograft bones, xenograft bones, bone powder, bone particles, bone fibers and a combination thereof.

18. The system of claim 2, wherein the flexible unit is combined with bone fusion enhancing substances selected from the group consisting of bone morphogenic proteins (BMPs), DNA vectors expressing BMPs and a combination thereof.

19. The system of claim 17, wherein the allograft bone, autograft bone, xenograft bone, bone powder, bone particles, and the bone fibers are selected from the group consisting of partially demineralized bone, fully demineralized bone, fully mineralized bone and a combination thereof.

20. The system of claim 6, wherein the at least one first fastener has a proximal and distal end, at least one of the proximal and distal ends and a portion of the flexible unit being tied, knotted, clamped, crimped, bonded, or glued together.

21. The system of claim 2, wherein the flexible unit includes at least one preformed loop having various dimensions and thickness to generate the corrective force.

22. The system of claim 20, wherein the proximal end of the at least one first fastener is provided with an enlargement configured to be looped around by the portion of the flexible unit.

23. The system of claim 2, further comprising a coupler configured to engage a portion of the flexible unit to prevent further displacement thereof upon generating the corrective force sufficient to stabilize the coupled bony members upon establishing the desired relationship therebetween, the coupler being made from a malleable or deformable material selected from the group consisting of a sheet of thin metal, plastic, bone, and a combination thereof.

24. The system of claim 14, wherein the at least one protrusion provided on the bearing surface of the support plate is selected from the group consisting of spikes, grooves, barbs, ridges, knurling, splines and a combination thereof and configured to engage the outer surface of the one bony member.

25. The system of claim 23, wherein the coupler is configured to have a key and a groove spaced apart and engaging one another to lock the flexible unit after the coupler has been deformed upon establishing the desirable relationship between the coupled bony members.

26. The system of claim 5, wherein the at least one second bony member has a throughgoing channel configured to receive a portion of the flexible unit so that the flexible unit generates the corrective force applied to the one and at least one second bony members.

27. The system of claim 26, further comprising at least one sleeve configured to be at least partially inserted into the throughgoing channel and surrounding the portion of the flexible unit to prevent excessive wear of the flexible unit.

28. The system of claim 27, wherein the at least one sleeve has a body and at least one shoulder extending laterally from the body, the at least one shoulder having a surface provided with protrusions or surface roughenings configured to engage an outer surface of the at least one second bony member.

29. The system of claim 2, wherein the flexible unit further includes a plurality of lengths of tethering material, at least some of the plurality of lengths being interconnected with one another.

30. The system of claim 1, wherein the plurality of bony members form multiple motion segments each including a pair of adjacent bony members and an intermediary member located therebetween, the system further comprising an auxiliary coupled to at least one motion segment and spaced from the flexible unit to generate an auxiliary force directed generally opposite to the corrective force of the flexible unit, which is attached to the at least one motion segment, and facilitating establishment of the desirable relationship between the adjacent bony members and the intermediary member of the at least one motion segment relative to one another.

31. The system of claim 30, wherein the auxiliary unit has at least one elongated support element extending along a longitudinal axis and attached to the at least one bony motion segment, the at least one elongated support element being provided with pieces of bone, or bone composites, which constitute either an entire length of the at least one support element or sections thereof.

32. The system of claim 31, wherein opposite ends of the at least one elongated support element are sectioned parallel, perpendicular, or at an angle to the longitudinal axis of the at least one elongated support element.

33. The system of claim 31, wherein the at least one support element has a textured surface including at least one attaching element selected from the group consisting of spikes, pyramidal protrusions, grooves and a combination thereof and configured to facilitate attachment of the auxiliary unit to the at least one bony member.

34. The system of claim 31, wherein the at least one bony motion segment includes two adjacent vertebral bodies juxtaposed with an intervertebral disc, the flexible unit and the auxiliary unit being attached directly to posterior elements of the bony motion segment to establish the desirable relationship between the adjacent vertebral bodies including an anatomically desired curvature or shape, wherein the posterior elements are selected from the group consisting of pedicles, lamina, facets, and transverse, spinous, costal, mammilary, accessory, inferior, superior processes and a combination thereof.

35. The system of claim 1, further comprising a restorative correcting element attached to at least one of the coupled bony members and configured to restore a desired anatomical shape thereof, the flexible unit being coupled so that to prevent the at least one restorative element from displacement upon establishing the desirable relationship between the coupled bony members.

36. The system of claim 35, wherein the flexible unit has a portion attached to the restorative correcting element.

37. The system of claim 35, wherein the restorative correcting element is made from a biocompatible material selected from the group consisting of metals, shape memory alloys, carbon graphite composites, polymers, alograft bones, autograft bones, xenograft bones, bone powder, bone particles, bone fibers and a combination thereof.

38. The system of claim 35, wherein the coupled bony members are spaced apart to form a space therebetween, the restorative correcting element having a body, which is configured to fit the space, and at least one projection extending laterally from the body and over at least one of the coupled bony members and being attached thereto by the flexible unit.

39. The system of claim 35, wherein the coupled bony members form at least one bony motion segment having a pair of bony portions and a non-bony intermediate portion, the restorative correcting element being selectively attached to the pair of bony and intermediate non-bony portions to restore the desirable relationship therebetween.

40. The system of claim 29, wherein the plurality of lengths of the flexible unit are selectively braided, woven, or tied together.

41. The system of claim 2, wherein the flexible unit is configured to have a pattern selected from the group consisting of a shoelace pattern, repeating loops, a crisscross pattern, a mesh-pattern, a linear array and a combination thereof.

42. The system of claim 1, wherein the coupled bony members constitute at least one bony motion segment selected from the group consisting of spine, finger, toe, hand, wrist, ankle, foot and a combination thereof.

43. The system of claim 6, wherein the flexible unit and the first and at least one second 10 fasteners are combined with a substance selected from the group of Phytochemical compounds, hydroxybenzoics compounds, flavonols compounds, turmeric curcuminare compounds, quercetin compounds, gallic acid, protocatechuic acid, chlorogenic acid, tamoxifin, 5-fluorouracil, matrix metalloproteinase inhibitors, TGFBeta inhibitors, Agaricus bisporus, tetrandrine, chitosan-polyvinyl pyrrolidone hydrogels and a combination thereof to provide scar inhibitoring treatments.

44. The system of claim 1, wherein the flexible unit includes at least one length thereof selected from the group consisting of metal wire, polymer strand and a combination thereof.

45. The system of claim 1, further comprising at least one rigid system coupled to a bony member located adjacent to the bony members coupled by the flexible unit, the rigid system including at least one rod, plate, screw, hook, rivet, cable, wire, and a combination thereof.

46. The system of claim 20, wherein at least one of the proximal and distal ends of the first fastener has an opening configured to receive the portion of the flexible unit.

47. A method for selectively stabilizing a plurality of bony members, the method comprising the steps of:
loading a plurality of bony members, thereby establishing a desirable relationship therebetween;
providing a flexible unit with a predetermined length, thickness, quantity and size; and
selectively coupling the flexible unit to the loaded bony members to generate a corrective force sufficient to maintain the load and to stabilize the coupled bony members relative to one another upon establishing the desirable relationship therebetween.

48. The method of claim 47, wherein the step of providing the flexible unit includes forming the flexible unit with a polygonal or circular cross-section from material selected from the group consisting of fascia, abdominal peritoneum, tendons, gracilis, iliotibial band, small intestine submucosa, perichondrial tissue, completely demineralized bone, partially demineralized bone, ligament, silk, collagen, elastin, reticulin, cellulose, and a combination thereof, the tethering material includes at least one length of the tethering material.

49. The method of claim 48, wherein the step of loading the plurality of bony members includes applying a compressive or tensile load, and the step of selectively coupling the flexible unit includes generating the corrective force within the flexible unit applied in a direction opposite to a direction of the load and including a compressive or tensile force.

50. The method of claim 48, wherein the step of providing the flexible unit including fascia, abdominal peritoneum, tendons, gracilis, iliotibial band, small intestine submucosa, perichondrial tissue, completely demineralized bone, partially demineralized bone, and ligament includes making the flexible unit from a substance selected from the group consisting of autograft flexible unit, an allograft flexible unit, a xenograft flexible unit, and a combination thereof.

51. The method of claim 50, further comprising the step of combining the substance with bone fusion enhancers selected from the group consisting of bone morphogenic proteins (BMPs), DNA vectors expressing BMPs and a combination thereof to form the flexible unit.

52. The method of claim 48, further comprising the step of braiding, weaving, knotting, looping, crocheting, knitting, gluing, welding, crimping, or molding the material used to form the flexible unit.

53. The method of claim 49, wherein the coupled bony members constitute at least one bony motion segment including a pair of vertebral bodies separated by an intervertebral disc or motion members of fingers, toes, hands, wrists, feet, or ankles separated by an intermediary member, the flexible unit being coupled to generate the corrective force sufficient to stabilize the coupled bony members so that the at least one bony motion segment has a desirable anatomical curvature or shape while not affecting mobility of non-coupled bony members.

54. The method of claim 49, further comprising the steps of providing the coupled bony members each with a channel dimensioned to receive a portion of the flexible unit, inserting a sleeve within the passage, guiding a portion through the sleeve and attaching opposite ends of the flexible unit together to generate the corrective force sufficient to establish the desirable relationship between the coupled bony members.

55. The method of claim 49, further comprising the step of coupling a portion of the flexible unit to a first fastener and attaching the first fastener to a respective bony member, and coupling a second portion of the flexible unit to an at least one second fastener spaced from the first fastener and attached to a respective bony member so that the flexible unit generates the corrective force sufficient to provide the coupled bony members with the desirable relationship.

56. The method of claim 55, further comprising engaging the portion of the flexible unit between a shank of the first fastener and a peripheral wall of a recess formed in the respective bony member upon introducing the first fastener therein, thereby preventing displacement of the flexible unit upon generating the corrective force.

57. The method of claim 55, wherein the portion of the flexible unit is looped around a proximal end of the first fastener or threaded though an eyelet provided on the proximal end and coupled to another portion of the flexible unit to form an endless flexible unit.

58. The method of claim 55, wherein the first and at least one second fasteners are selected from the group consisting of screws, wedges, suture anchors, wedge buttons, clips, snaps, friction fittings, compressive fittings, expanding rivets, staples, nails, crimps and a combination thereof, the first and at least one second fasteners being coupled to the bony members detachably or permanently.

59. The method of claim 55, wherein the flexible unit coupled to the bony members has a pattern selected from a shoelace pattern, a criss-cross pattern, repeated loops, a linear array, a mesh-pattern and a combination thereof.

60. The method of claim 55, further comprising the step of providing a support plate attached to one of the coupled bony members by the first fastener and configured to have an inner surface juxtaposed with and conforming to an outer surface of the one bony member, and directly attaching the portion of the flexible unit to the at least one protrusion.

61. The method of claim 60, wherein the support plate and the first fastener are integrally made to form a unitary assembly, the method further comprising the step of providing an outer surface of the support plate with tethering posts configured to receive the portion of the flexible unit.

62. The method of claim 53, further comprising the steps of utilizing an auxiliary system operative to apply the load to the one bony motion segment before applying the corrective force, the auxiliary system being mounted on the bony motion segment at a distance from the flexible, and utilizing a rigid system configured to correct a relationship of another bony motion segment located adjacent to the bony motion segment exposed to the corrective force.

63. The method of claim 47, wherein the plurality of bony members constitute a singular body, the method further comprising coupling an artificial implant to the singular body, thereby restoring the desirable relationship between the plurality of bony members, and attaching the flexible unit to the bony members to apply the corrective force so that the implant is stabilized relative to the singular body.

64. A kit for stabilizing multiple bony members comprising a flexible stabilizing assembly including:
at least one fastener configured to be coupled to at least one bony member;
an inserter instrument operative to introduce the at least one fastener to the at least one bony motion member; and
a plurality of flexible units each configured to have a predetermined size, dimension and quantities selected to apply and maintain a desired corrective force to the at least one bony motion member upon attachment a respective flexible unit to the at least one bony motion member.

65. The kit of claim 64, wherein the flexible unit is selected from a group consisting of a plate-like element, a weave, a strand, and at least one rope-like element.

66. The kit of claim 65, wherein the plate-like element is configured to extend between and stabilize at least two bony motion members.

67. The kit of claim 65, further comprising at least one rigid support plate configured to be coupled to the at least one bony member by the at least one fastener, whereas the fastener is integrally formed with or detachably coupled to the at least one support plate.

68. The kit of claim 67, wherein the support plate has at least one spike configured to deform so as to secure the plate to the at least one bony motion member.

69. The kit of claim 65, wherein the plate-like element, weave, strand, and ropelike element is made from material selected from the group consisting of metal, wire, fascia, abdominal peritoneum, tendons, gracilis, iliotibial band, small intestine submucosa, perichondrial tissue, completely demineralized bone, partially demineralized bone, ligament, silk, collagen, elastin, reticulin, cellulose, non-resorbable polymers and a combination thereof.

70. The kit of claim 64, further comprising a rigid system configured to provide multiple bony members located adjacent to the at least one bony segment with a desirable anatomical relationship, the rigid system including at least one rod, plate, screw, hook, rivet, wire, cable and a combination thereof.

71. The kit of claim 64, further comprising a group of restoring elements having various shapes and dimensions and configured to restore a desirable anatomical relationship of the at least one bony motion member.

72. The kit of claim 64, further comprising an auxiliary system configured to couple to and apply a load to the bony members sufficient to establish a desirable relationship therebetween further maintained by the corrective force, which is generated by a respective flexible unit.

73. The kit of claim 72, wherein the auxiliary system has at least one elongated support element extending along a longitudinal axis, the at least one elongated support element being provided with pieces of bone, or bone composites, which constitute either an entire length of the at least one support element or sections thereof, and with opposite ends sectioned parallel, perpendicular, or at an angle to the longitudinal axis.
